(19) United States
(12) Patent Application Publication
(10) Pub. No.: US 2013/0053913 A1
(43) Pub. Date: Feb. 28, 2013

(54) Method and System to Adjust Pacing Parameters Based on Systolic Interval Heart Sounds

(71) Applicants: Steve Koh, South Pasadena, CA (US); Gene A. Bornzin, Simi Valley, CA (US); Jeffery D. Snell, Chatsworth, CA (US)

(72) Inventors: Steve Koh, South Pasadena, CA (US); Gene A. Bornzin, Simi Valley, CA (US); Jeffery D. Snell, Chatsworth, CA (US)

(73) Assignee: PACESETTER, INC., Sylmar, CA (US)

(21) Appl. No.: 13/223,117

(22) Filed: Aug. 31, 2011

(51) Int. Cl. A61N 1/368

(52) U.S. Cl. 607/17

(57) ABSTRACT

A method is provided to determine pacing parameters for an implantable medical device (IMD) and collects heart sounds during the cardiac cycles. The method comprises changing a value for a pacing parameter between the cardiac cycles and analyzing a characteristic of interest from the heart sounds. The method comprises setting a desired value for the pacing parameter based on the characteristic of interest from the heart sounds. The system comprises inputs configured to be coupled to at least one lead having electrodes to sense intrinsic events and to deliver pacing pulses over cardiac cycles. The system has a sensor for collecting heart sounds during cardiac cycles and controller to control delivery of pacing pulses based on pacing parameters. The controller changes a value for at least one of the pacing parameters between the cardiac cycles and provides an analysis module to analyze a characteristic of interest from the heart sounds.

### BACKGROUND OF THE INVENTION

Embodiments of the subject matter described herein generally relate to adjustment of parameters for implantable medical devices, and more particularly to adjusting pacing parameters based on heart sounds to improve hemodynamic performance.

An implantable medical device (IMD) is implanted in a patient to monitor, among other things, electrical activity of a heart and to deliver appropriate electrical therapy, as required. Implantable medical devices include pacemakers, cardioverters, defibrillators, implantable cardioverter defibrillators (ICD), and the like. The electrical therapy produced by an IMD may include pacing pulses, cardioverting pulses, and/or defibrillator pulses to reverse arrhythmias (e.g., tachycardias and bradycardias) or to stimulate the contraction of cardiac tissue (e.g., cardiac pacing) to return the heart to its normal sinus rhythm. These pulses are referred to as stimulus or stimulation pulses.

IMDs supply a pacing therapy to hearts to treat various arrhythmias. The pacing therapy may include supplying stimulus pulses to the left and/or right ventricles of the heart at a programmed stimulation rate when intrinsic events are not detected within certain time periods, often referred to as delays. Applying the stimulus pulses to the ventricles may restore mechanical synchrony to the heart. For example, the stimulus pulses may return the heart to a normal rate of ventricular contraction.

Pacing therapies of some known IMDs monitor cardiac events in certain chambers of the heart to determine when to supply stimulus pulses to other chambers of the heart. For example, after detecting a paced or intrinsic cardiac event in the right atrium (or right ventricle), the IMD monitors the ventricles (or left ventricle) for cardiac signals to determine if a subsequent intrinsic cardiac event occurs during a predetermined delay after the preceding cardiac event. Examples of the delays include a delay between an atrial event and the successive ventricular event (AV delay), a delay between a right ventricular event and a left ventricular event (W delay), and a delay between a ventricular event and the next atrial event (VA delay). The AV delay, VV delay and VA delay are examples of some of the pacing parameters that are programmable by a clinician, and in certain types of IMDs are automatically adjusted during operation. If no subsequent cardiac event is detected during the predetermined delay, then the IMD supplies a stimulus pulse. When responding to an atrial cardiac event, the IMD will supply pulses to one or both ventricles of the heart to induce contraction of the heart. When responding to a right ventricular cardiac event, the IMD will supply pulses to the left ventricle or right atrium.

The pacing parameters are adjusted in an effort to improve hemodynamic performance of an individual patient. For example, when the AV delay is too short, a patient may experience reduced cardiac output. However, when the AV delay is properly set, the patient experiences good cardiac output (relative to the patients overall health) and good overall hemodynamic performance that may be as good as possible.

Recently, it has been proposed to utilize heart sounds in connection with certain aspects of IMD operation. Heart sounds are the noises generated by the beating heart and the resultant flow of blood, and are typically referred to as S1, S2, S3 and S4. An S1 heart sound is caused by the sudden block of reverse blood flow due to closure of the atrioventricular valves (mitral and tricuspid) at the beginning of ventricular contraction. When the ventricles begin to contract, so do the papillary muscles in each ventricle. The papillary muscles are attached to the tricuspid and mitral valves via chorda tendinae, which bring the cusps of the valve closed (chorda tendinae also prevent the valves from blowing into the atria as ventricular pressure rises due to contraction). The closing of the inlet valves prevents regurgitation of blood from the ventricles back into the atria. The S1 sound results from reverberation within the blood associated with the sudden block of flow reversal by the valves.

An S2 heart sound is caused by the sudden block of reversing blood flow due to closure of the aortic valve and pulmonary valve at the end of ventricular systole, i.e beginning of ventricular diastole. As the left ventricle empties, its pressure falls below the pressure in the aorta, aortic blood flow quickly reverses back toward the left ventricle, catching the aortic valve leaflets and is stopped by aortic (outlet) valve closure. Similarly, as the pressure in the right ventricle falls below the pressure in the pulmonary artery, the pulmonary (outlet) valve closes. The S2 sound results from reverberation within the blood associated with the sudden block of flow reversal.

Heretofore, it has been proposed to control an AV interval or VV interval based on a pulse width of an “S1” heart sound. It also has been proposed to control the AV interval or VV interval based on a sum of the duration of the S1 heart sound and an “S2” heart sound.

However, a need remains to identify better techniques for monitoring hemodynamic performance and to control adjustment of various parameters.

### SUMMARY

In accordance with one embodiment, a method is provided to determine pacing parameters for an implantable medical device (IMD). The method comprises an implantable medical device (IMD) collecting heart sounds during the cardiac cycles. The heart sounds include sounds representative of a degree of blood flow turbulence wherein the heart sounds include S1, S2 and linking segments. The S1 segment is associated with initial systole activity, the S2 segment is associated with initial diastole activity and the linking segment is associated with heart activity occurring during a systolic interval between the initial systole and diastole activity.

The method further comprises changing a value for a pacing parameter between the cardiac cycles and analyzing a characteristic of interest from the heart sounds within at least a portion of the linking segment. The characteristic of interest is indicative of an amount of heart sounds over at least a portion of the systolic interval between the initial systole and diastole activity, the level of the characteristic of interest changing as the pacing parameter is changed. The method further provides setting a desired value for the pacing parameter based on the characteristic of interest from the heart sounds from the linking segment.

Optionally, the method provides an analyzing operation that includes identifying S1 and S2 peaks associated with the initial systole and diastole activity, respectively, and integrates the heart sounds over the time period between the S1 and S2 peaks. Additionally, the method further provides an analyzing operation that determines an energy content within the linking segment, the energy content within the linking segment excluding an energy content within the S1 and S2 segments and the setting operation reducing the energy content within the linking segment to below a predetermined level.

The analyzing operation further determines S1 energy content associated with the S1 segment, S2 energy content associated with the S2 segment, and linking energy content associated with the linking segment. The S1, S2 and linking energy contents may be mutually exclusive of one another. The setting operation limits a ratio of the S1, S2 and linking energy contents to a predetermined level.

Optionally, the method provides that the characteristic analyzed during the analyzing operation identifies at least one of intensity or energy content of the heart sounds as the amount over an entirety of the systolic interval following the S1 heart sound. The method further comprises determining a minimum level for the heart sounds from a collection of the heart sounds collected over multiple cardiac cycles, the setting operation setting the desired value to correspond to the minimum level for the heart sounds. Optionally, the collecting operation may be performed during implantation of the IMD wherein an external programmer controls the collecting, changing and analyzing operations.

The collecting operation may include deriving heart sounds from signals produced by an accelerometer within the IMD. The IMD may represent a rate-responsive IMD. The collecting, changing and identifying operations are repeated periodically by the rate-responsive IMD to provide real-time updates to the pacing parameter throughout operation.

Additionally, the pacing parameter may represent at least one of an AV delay, a VV delay and a VA delay. The changing operation changes at least one of the AV delay, the W delay and VA delay in order reduce systolic turbulence and regurgitation.

In accordance with an embodiment, a system is provided that comprises inputs configured to be coupled to at least one lead having electrodes to sense intrinsic events and to deliver pacing pulses over cardiac cycles. The system may include an IMD and/or a programmer. The system has a sensor for collecting heart sounds during cardiac cycles. The heart sounds include sounds representative of a degree of blood flow turbulence. The sensor collects the heart sounds that include S1, S2 and linking segments. The S1 segment is associated with initial systole activity, the S2 segment associated with initial diastole activity. The linking segment is associated with heart activity occurring during a systolic interval between the initial systole and diastole activity.

The system further provides a controller to control delivery of pacing pulses based on pacing parameters. The controller changes a value for at least one of the pacing parameters between the cardiac cycles. Additionally, the system comprises an analysis module to analyze a characteristic of interest from the heart sounds within at least a portion of the linking segment. The characteristic of interest is indicative of an amount of the heart sounds over at least a portion of the systolic interval between the initial systole and diastole activity. The level of the characteristic of interest changes as the pacing parameter is changed. A setting module sets a desired value for the pacing parameter based on the characteristic of interest from the heart sounds from the linking segment.

Optionally, the analysis module identifies S1 and S2 peaks associated with the initial systole and diastole activity, respectively, and integrates the heart sounds over the time period between the S1 and S2 peaks. The analysis module may determine an energy content within the linking segment. The energy content within the linking segment excludes an energy content within the S1 and S2 segments. The setting operation may reduce the energy content within the linking segment to below a predetermined level.

Optionally, the analysis module may determine S1, S2 and linking energy contents individually associated with the S1, S2 and linking segments, respectively, where the S1, S2 and linking energy contents are mutually exclusive of one another. The setting module may limit a ratio of the S1, S2 and linking energy contents to a predetermined level.

The analysis module may identify at least one of intensity or energy content as the amount of the heart sounds over an entirety of the systolic interval following the S1 heart sound.

In accordance with an embodiment, an IMD is provided that uses a device-based accelerometer to measure heart sound intensity to assess the degree of blood turbulence during systolic (ejection) as well as diastolic (filling) time for a set of cardiac device therapy parameters. A desired parameter value may be chosen based on minimal heart sound intensity between S1 and S2.

Further, embodiments can be implemented to apply in rate adaptive pacing where AV delay adaptation is desired. On-the-fly AV delay adaptation is possible to achieve relatively low systolic turbulence.

In accordance with an embodiment, a PSA/Programmer based system is provided with a wand having mean of acoustic sensing (microphone or accelerometer) and that will determine optimal parameters during CRT device follow-ups or at the time of implant.

### BRIEF DESCRIPTION OF THE DRAWINGS

FIG. 1 illustrates an implantable medical device that is formed in accordance with an embodiment of the present invention.

FIG. 2 illustrates a block diagram of exemplary internal segments of the IMD.

FIG. 3 illustrates a relationship, during a single cardiac cycle, between exemplary left ventricular pressure, aortic pressure, left atrial pressure, heart sounds, left ventricular volume and intracardiac electrogram signals.

FIG. 4 illustrates a functional block diagram of an external programmer that is implemented in accordance with one example embodiment.

FIG. 5 illustrates a distributed processing system in accordance with one embodiment.

FIG. 6 illustrates a chart showing hypothetical examples of how heart sound measurements, over certain portions of the cardiac cycle, may relate to different values for AV delay.

FIG. 7 illustrates a hypothetical contractility behavior plotting a potential relation between AV delay (along the horizontal axis) and maximum left ventricular pressure per unit time (maxLVdp_dt) during a cardiac cycle.

FIG. 8 illustrates one method for determining a value of at least one pacing parameter in accordance with an embodiment.

FIG. 9 illustrates exemplary heart sounds collected over a single cardiac cycle or composite heart sounds combined from an ensemble of cardiac cycles.

FIG. 10 illustrates a processing sequence for identifying S1 and S2 heart sounds for a single cardiac cycle or ensemble of cardiac cycles and analyzing the heart sounds for a characteristic of interest.

FIG. 11 illustrates an alternative process to analyze a characteristic of interest from the heart sounds.

FIG. 12 illustrates a set of histograms that may be created from one cardiac cycle or from an ensemble of cardiac cycles.

### DETAILED DESCRIPTION

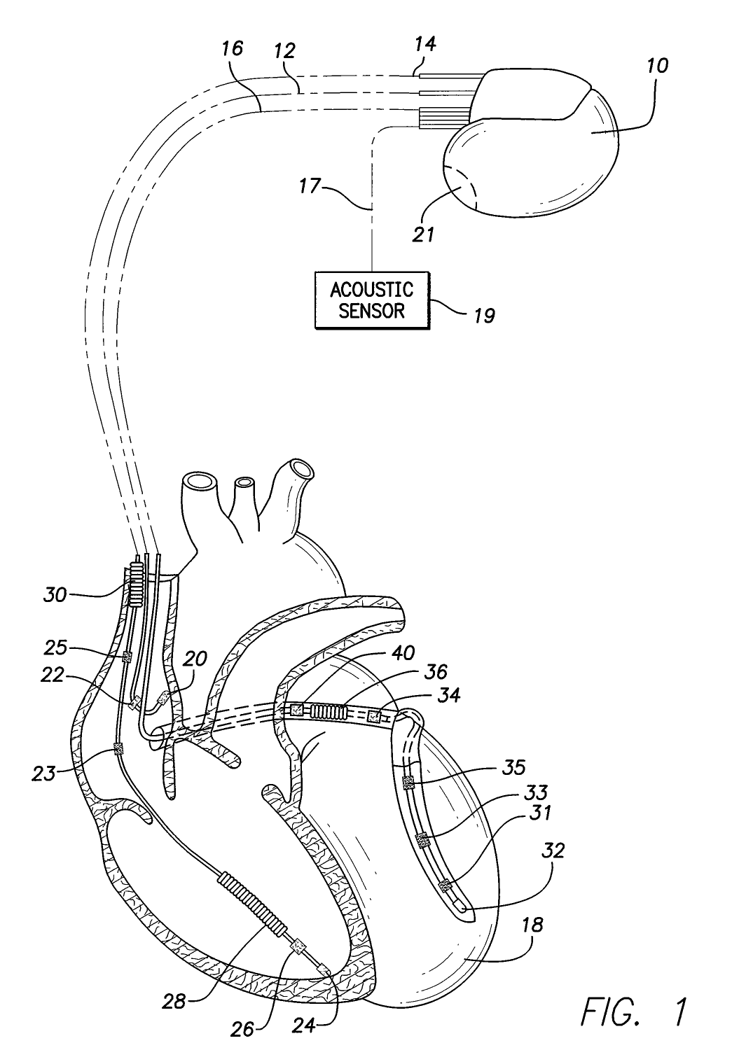

FIG. 1 illustrates an IMD 10 that is formed in accordance with an embodiment of the present invention. The IMD 10 is connected to various leads, such as right atrial lead 12, right ventricular lead 14, and coronary sinus lead 16. Optionally, more or fewer leads may be used, as well as different configurations of leads. The IMD 10 may be a dual-chamber stimulation device capable of treating both fast and slow arrhythmias with stimulation therapy, including cardioversion, defibrillation, and pacing stimulation, as well as capable of detecting heart failure, evaluating its severity, tracking the progression thereof, and controlling the delivery of therapy and warnings in response thereto. The atrial lead 12 has an atrial tip electrode 20 and an atrial ring electrode 22 implanted in the atrial appendage. The ventricular lead 14 has a ventricular tip electrode 24, a right ventricular ring electrode 26, a right ventricular (RV) coil electrode 28, and a superior vena cava (SVC) coil electrode 30. The ventricular lead 14 is capable of receiving cardiac signals, and delivering stimulation in the form of pacing and shock therapy to the right ventricle.

The “coronary sinus” lead 16 is placed in the “coronary sinus region” via the coronary sinus for positioning a distal electrode adjacent to the left ventricle and/or additional electrode(s) adjacent to the left atrium. The coronary sinus lead 16 includes a left ventricular tip electrode 32, a left atrial ring electrode 34, and a left atrial coil electrode 36. Optionally, the lead 16 may also include multiple LV electrodes 31, 32, 33 and 35 to afford additional left ventricular sensing and pacing sites. It should also be understood that fewer or additional stimulation leads (with one or more pacing, sensing and/or shocking electrodes) may be used in order to efficiently and effectively provide pacing stimulation to the left side of the heart or atrial cardioversion and/or defibrillation.

One or more of the leads 12, 14 and 16 detect intracardiac electrogram (IEGM) signals that form an electrical activity indicator of myocardial function over multiple cardiac cycles. The IEGM signals represent analog signals that are subsequently digitized and analyzed to identify waveforms of interest. Examples of waveforms identified from the IEGM signals include the P-wave, T-wave, the R-wave, the QRS complex and the like. The lead 16 may include a sensor 40 for sensing left atrial activity.

The IMD 10 may be coupled to an acoustic sensor 19 through an insulated conductor 17. As shown in FIG. 1, the acoustic sensor 19 is positioned proximate and external to the heart. Optionally, the acoustic sensor 19 may be located internal to the IMD 10 (shown as acoustic sensor 21). Optionally, an acoustic sensor 23 may be provided on one or more of leads 12, 14 and 16. The acoustic sensors 23 may be provided in or near the aorta, or in or near any chamber of the heart from which heart sounds are of interest. The acoustic sensors 19, 21 and/or 23 detect heart sounds, which represent an acoustic indicator of myocardial function.

The IMD 10 stores heart sound data sets over multiple cardiac cycles, continuously or periodically (e.g., every hour, every day, etc.). The heart sound data sets may be analyzed by the IMD 10, or transmitted externally for analysis, such as by a programmer, a hospital network, a workstation and the like. The systolic and diastolic intervals may be determined from several indicators, such as IEGM, ECG, heart sounds, myocardial pressure and the like.

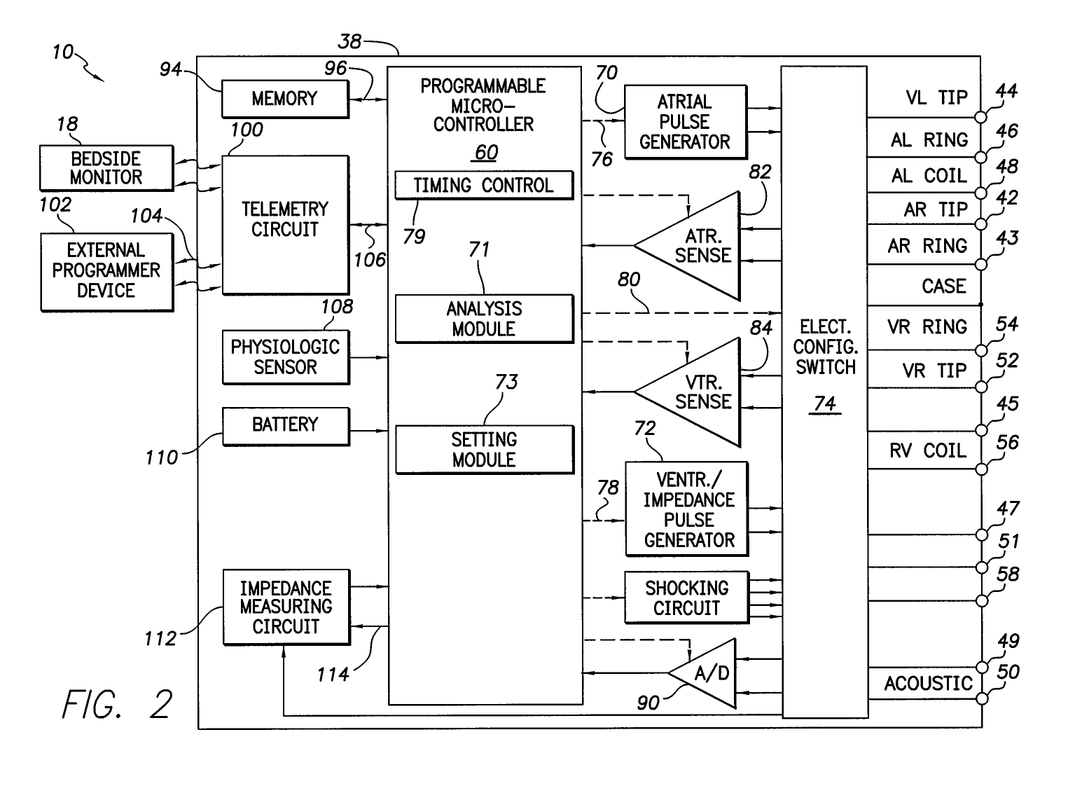

FIG. 2 illustrates a block diagram of exemplary internal components of the IMD 10. The IMD 10 is for illustration purposes only, and it is understood that the circuitry could be duplicated, eliminated or disabled in any desired combination to provide a device capable of treating the appropriate chamber(s) with cardioversion, defibrillation and/or pacing stimulation as well as providing for apnea detection and therapy. The housing 38 for IMD 10, shown schematically in FIG. 2, is often referred to as the “can”, “case” or “case electrode” and may be programmably selected to act as the return electrode for all “unipolar” modes. The housing 38 may further be used as a return electrode alone or in combination with one or more of the coil electrodes, 36, 28 and 30, for shocking purposes. The housing 38 further includes a connector (not shown) having a plurality of terminals, 42-52, 54, 56 and 58 (shown schematically and, for convenience, the names of the electrodes to which they are connected are shown next to the terminals). A right atrial tip terminal (A.sub.R TIP) 42 is adapted for connection to the atrial tip electrode 20 and a right atrial ring (A.sub.R RING) terminal 43 is adapted for connection to right atrial ring electrode 22. A left ventricular tip terminal (V.sub.L TIP) 44, a left atrial ring terminal (A.sub.L RING) 46, and a left atrial shocking terminal (A.sub.L COIL) 48 are adapted for connection to the left ventricular ring electrode 32, the left atrial tip electrode 34, and the left atrial coil electrode 36, respectively. A right ventricular tip terminal (V.sub.R TIP) 52, a right ventricular ring terminal (V.sub.R RING) 54, a right ventricular shocking terminal (R.sub.V COIL) 56, and an SVC shocking terminal (SVC COIL) 58 are adapted for connection to the right ventricular tip electrode 24, right ventricular ring electrode 26, the RV coil electrode 28, and the SVC coil electrode 30, respectively.

An acoustic terminal 50 is adapted to be connected to the external acoustic sensor 19 or 23 or the internal acoustic sensor 21, depending upon which (if any) of sensors 19, 21 and 23 is used. Terminal 51 is adapted to be connected to sensor 25 to collect measurements associated with glucose levels, natriuretic peptide levels, or catecholamine levels.

The IMD 10 includes a programmable microcontroller 60, which controls operation. The microcontroller 60 (also referred to herein as a processor module or unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by program code stored in memory. The details of the design and operation of the microcontroller 60 are not critical to the invention. Rather, any suitable microcontroller 60 may be used that carries out the functions described herein. Among other things, the microcontroller 60 receives, processes, and manages storage of digitized cardiac data sets from the various sensors and electrodes. For example, the cardiac data sets may include IEGM data, pressure data, heart sound data, and the like.

The IMD 10 includes an atrial pulse generator 70 and a ventricular/impedance pulse generator 72 to generate pacing stimulation pulses for delivery by the right atrial lead 12, the right ventricular lead 14, and/or the coronary sinus lead 16 via an electrode configuration switch 74. It is understood that in order to provide stimulation therapy in each of the four chambers of the heart, the atrial and ventricular pulse generators, 70 and 72, may include dedicated, independent pulse generators, multiplexed pulse generators or shared pulse generators. The pulse generators, 70 and 72, are controlled by the microcontroller 60 via appropriate control signals, 76 and 78, respectively, to trigger or inhibit the stimulation pulses.

The microcontroller 60 further includes timing control circuitry 79 used to control the timing of such stimulation pulses (e.g., pacing rate, atria-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, and the like. Switch 74 includes a plurality of switches for connecting the desired electrodes to the appropriate I/O circuits, thereby providing complete electrode programmability. Accordingly, the switch 74, in response to a control signal 80 from the microcontroller 60, determines the polarity of the stimulation pulses (e.g., unipolar, bipolar, etc.) by selectively closing the appropriate combination of switches (not shown) as is known in the art.

Atrial sensing circuit 82 and ventricular sensing circuit 84 may also be selectively coupled to the right atrial lead 12, coronary sinus lead 16, and the right ventricular lead 14, through the switch 74 for detecting the presence of cardiac activity in each of the four chambers of the heart. Accordingly, the atrial (ATR SENSE) and ventricular (VTR SENSE) sensing circuits, 82 and 84, may include dedicated sense amplifiers, multiplexed amplifiers or shared amplifiers. The outputs of the atrial and ventricular sensing circuits, 82 and 84, are connected to the microcontroller 60 which, in turn, are able to trigger or inhibit the atrial and ventricular pulse generators, 70 and 72, respectively, in a demand fashion in response to the absence or presence of cardiac activity in the appropriate chambers of the heart.

Cardiac signals are also applied to the inputs of an analog-to-digital (A/D) data acquisition system 90. The data acquisition system 90 is configured to acquire IEGM signals, convert the raw analog data into a digital IEGM signal, and store the digital IEGM signals in memory 94 for later processing and/or telemetric transmission to an external device 102. The data acquisition system 90 is coupled to the right atrial lead 12, the coronary sinus lead 16, and the right ventricular lead 14 through the switch 74 to sample cardiac signals across any combination of desired electrodes. The data acquisition system 90 is also coupled, through switch 74, to one or more of the acoustic sensors 19, 21 and 23. The data acquisition system 90 acquires, performs ND conversion, produces and saves the digital pressure data, and/or acoustic data.

The controller 60 controls the acoustic sensor 19 and/or physiologic sensor 108 to collect heart sounds during one or more cardiac cycles. The heart sounds include sounds representative of a degree of blood flow turbulence. The sensor 19 or 108 collects the heart sounds that include S1, S2 and linking segments. The S1 segment is associated with initial systole activity. The S2 segment is associated with initial diastole activity. The linking segment is associated with at least a portion of heart activity occurring between the S1 and S2 segments during a systolic interval between the initial systole and diastole activity. The controller 60 changes a value for at least one of the pacing parameters between the cardiac cycles. The controller 60 implements one or more processes described herein to determine values for one or more pacing parameters that yield a desired level of hemodynamic performance.

The controller 60 includes an analysis module 71 and a setting module 73 that function in accordance with embodiments described herein. The analysis module 71 analyzes a characteristic of interest from the heart sounds within at least a portion of the linking segment. The characteristic of interest is indicative of an “amount” of the heart sounds over at least a portion of the systolic interval between the initial systole and diastole activity. The amount of the heart sounds may be derived in different manners, such as determining the energy content, intensity and the like, as well as relations therebetween. The level of the characteristic changes as the pacing parameter is changed. The setting module 73 sets a desired value for the pacing parameter based on the characteristic of interest from the heart sounds for at least the portion of the linking segment. The pacing parameter may represent at least one of an AV delay, a VV delay, a VA delay, intra-ventricular delays, electrode configurations and the like. The controller 60 changes at least one of the AV delay, the VV delay, the VA delay, the intra-ventricular delays, electrode configurations and like in order to reduce systolic turbulence and regurgitation.

By way of example, with reference to FIG. 1, the controller 60 may utilize different combinations of the electrodes on the lead 16 (as the change in pacing parameters) to deliver different pacing stimulus when analyzing the characteristic of interest in the heart sounds (e.g., electrodes 31 and 35, or electrodes 31, 33 and 35, or electrodes 40, 35 and 32, or electrodes 31-36 and 40, etc.). As another example, the controller 60 may utilize different timing configurations associated with left ventricular sensing (as the change in pacing parameters) when analyzing the characteristic of interest in the heart sounds. For example, the timing configuration may assign one inter &&&

The analysis module 71 may identify S1 and S2 peaks associated with the initial systole and diastole activity, respectively, and integrate the heart sounds over the time period between the S1 and S2 peaks to derive the amount of the heart sounds. Optionally, the analysis module 71 may determine an energy content within the linking segment to derive the amount of the heart sounds. The energy content within the linking segment excludes an energy content within the S1 and S2 segments. The setting module reduces the energy content within the linking segment to below a predetermined level. Optionally, the analysis module 71 may determine S1, S2 and linking energy contents individually associated with the S1, S2 and linking segments respectively. In this example, the S1, S2 and linking energy contents are mutually exclusive of one another, and the setting module 73 limits a ratio of the S1, S2 and linking energy contents to a predetermined level. The characteristic of interest analyzed by the analysis module 71 may identify at least one of intensity or energy content of the heart sounds over an entirety of the systolic interval following the S1 heart sound. The analysis module 71 may determine a minimum level for the heart sounds from a collection of the heart sounds collected over multiple cardiac cycles. The setting module 73 may set the desired value to correspond to the minimum level for the heart sounds.

The microcontroller 60 is coupled to memory 94 by a suitable data/address bus 96, wherein the programmable operating parameters used by the microcontroller 60 are stored and modified, as required, in order to customize the operation of IMD 10 to suit the needs of a particular patient. The memory 94 also stores data sets (raw data, summary data, histograms, etc.), such as the IEGM data, heart sound data, pressure data, Sv02 data and the like for a desired period of time (e.g., 1 hour, 24 hours, 1 month). The memory 94 may store instructions to direct the microcontroller 60 to analyze the cardiac signals and heart sounds identify characteristics of interest and derive values for predetermined statistical parameters. The IEGM, pressure, and heart sound data stored in memory 94 may be selectively stored at certain time intervals, such as 5 minutes to 1 hour periodically or surrounding a particular type of arrhythmia of other irregularity in the heart cycle. For example, the memory 94 may store data for multiple non-consecutive 10 minute intervals.

The pacing and other operating parameters of the IMD 10 may be non-invasively programmed into the memory 94 through a telemetry circuit 100 in telemetric communication with the external device 102, such as a programmer, trans-telephonic transceiver or a diagnostic system analyzer, or with a bedside monitor 18. The telemetry circuit 100 is activated by the microcontroller 60 by a control signal 106. The telemetry circuit 100 allows intra-cardiac electrograms, pressure data, acoustic data, Sv02 data, and status information relating to the operation of IMD 10 (as contained in the microcontroller 60 or memory 94) to be sent to the external device 102 through an established communication link 104.

The memory 94 may be programmed with multiple conditions that, when satisfied by the indicators, are representative of potential ischemic episodes. For example, the conditions may include one or more of i) amplitudes and/or durations for heart sounds S1, S2, S3 and/or S4, ii) timing, intervals between and/or deviation of events of interest (e.g., mitral valve closing, mitral valve opening, aortic valve closing, aortic valve opening), iii) amplitudes and durations of points in the IEGM signal, and iv) durations of systolic interval, diastolic interval, isovolumic relaxation interval, and/or isovolumic contraction interval. The conditions may be preprogrammed from an external device or automatically set by the IMD 10 based on prior operation and historic data collected from the patient.

The IMD 10 may include an accelerometer or other physiologic sensor 108, commonly referred to as a “rate-responsive” sensor because it is typically used to adjust pacing stimulation rate according to the exercise state of the patient. Optionally, the physiological sensor 108 may further be used to detect changes in cardiac output, changes in the physiological condition of the heart, or changes in activity (e.g., detecting sleep and wake states) and to detect arousal from sleep. While shown as being included within IMD 10, it is to be understood that the physiologic sensor 108 may also be external to IMD 10, yet still be implanted within or carried by the patient. A common type of rate responsive sensor is an activity sensor incorporating an accelerometer or a piezoelectric crystal, which is mounted within the housing 38 of IMD 10.

The physiologic sensor 108 may be used as the acoustic sensor 23 (FIG. 1) that is configured to detect the heart sounds. For example, the physiologic sensor 108 may be an accelerometer that is operated to detect acoustic waves produced by blood turbulence and vibration of the cardiac structures within the heart 18 (e.g., valve movement, contraction and relaxation of chamber walls and the like). When the physiologic sensor 108 operates as the acoustic sensor 23, it may supplement or replace entirely acoustic sensors 19 and 21. Other types of physiologic sensors are also known, for example, sensors that sense the oxygen content of blood, respiration rate and/or minute ventilation, pH of blood, ventricular gradient, etc. However, any sensor may be used which is capable of sensing a physiological parameter that corresponds to the exercise state of the patient and, in particular, is capable of detecting arousal from sleep or other movement.

The IMD 10 additionally includes a battery 110, which provides operating power to all of the circuits shown. The IMD 10 is shown as having an impedance measuring circuit 112 which is enabled by the microcontroller 60 via a control signal 114. Herein, impedance is primarily detected for use in evaluating ventricular end diastolic volume (EDV) but is also used to track respiration cycles. Other uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 120 is advantageously coupled to the switch 74 so that impedance at any desired electrode may be obtained.

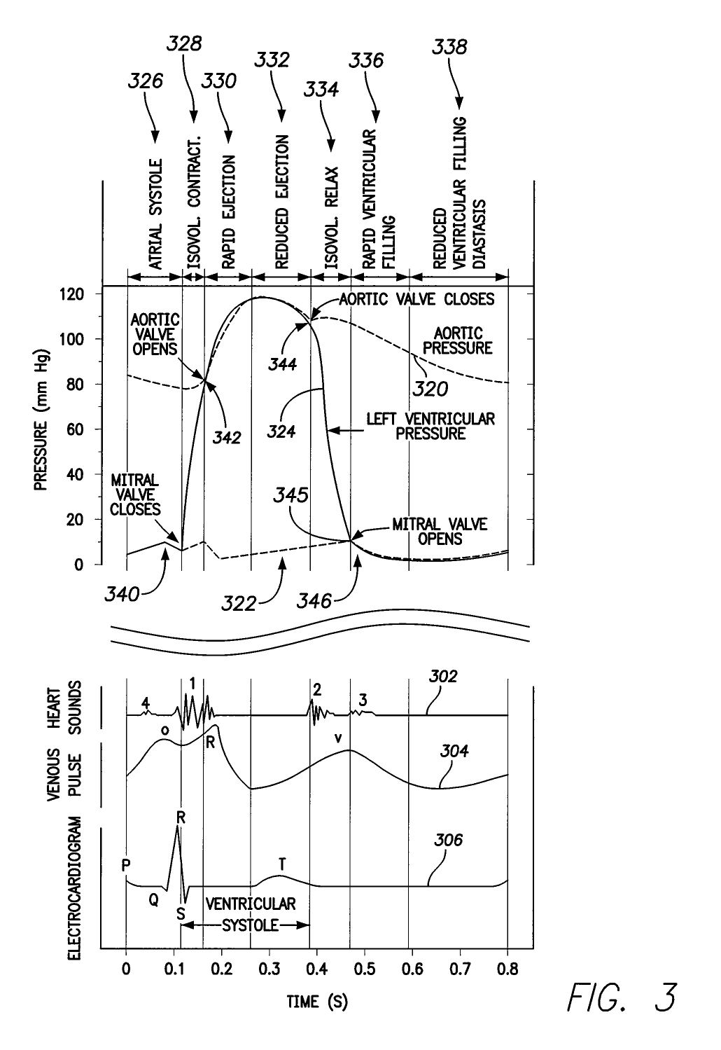

FIG. 3 illustrates a relationship, during a single cardiac cycle, between exemplary (not actual) left ventricular pressure (LVP) 324, aortic pressure (AP) 320, left atrial pressure (LAP) 322, heart sounds 302, left ventricular volume 304 and intracardiac electrogram (IEGM) signals 306. The signals of FIG. 3 have been divided into seven functional phases, namely atrial systole 326, isovolumic contraction 328 (ICD), rapid ejection 330, reduced ejection 332, isovolumic relaxation 334 (ISRD), rapid ventricular filling 336 and reduced ventricular filling diastasis 338. Certain functional phases are separated by particular events that are detectable. For example, the atrial systole 326 ends and isovolumic contraction 328 begins at the time of closure of the mitral valve (as denoted by event 340). The isovolumic contraction 328 ends when the aortic valve opens (as denoted by event 342). The isovolumic relaxation 334 begins at the time of closure of the aortic valve (as denoted by event 344), and the isovolumic relaxation 334 ends when the mitral valve opens (as denoted by event 346). Dashed lines 320 and 322 denote exemplary, not actual, aortic pressure and left atrial pressure responses over a cardiac cycle. The solid line 324 illustrates an exemplary, not actual, left ventricular pressure over the same cardiac cycle to illustrate a relation between left ventricular pressure 324 and aortic and left atrial pressure responses 320 and 322.

The heart sounds 302 (also referred to as a phonocardiogram) may be detected by the physiologic sensor 108 and/or the separate acoustic sensors 19 or 21. The sounds are produced by blood turbulence and vibration of cardiac structures due to the closing of the valves within the heart. Four sounds that may be identified are S1, S2, S3, and S4. S1 is usually the loudest heart sound and is the first heart sound during ventricular contraction. S1 occurs at the beginning of ventricular systole interval and represents the initial systole activity as it relates to the closure of the atrioventricular valves between the atria and the ventricles. S2 occurs at the beginning of the diastole interval and represents the initial diastole activity as it relates to the closing of the semilunar valves separating the aorta and pulmonary artery from the left and right ventricles, respectively. S3 occurs in the early diastolic period and is caused by the ventricular wall distending to the point it reaches its elastic limit. S4 occurs near the end of atrial contraction and is also caused by the ventricular wall distending until it reaches its elastic limit.

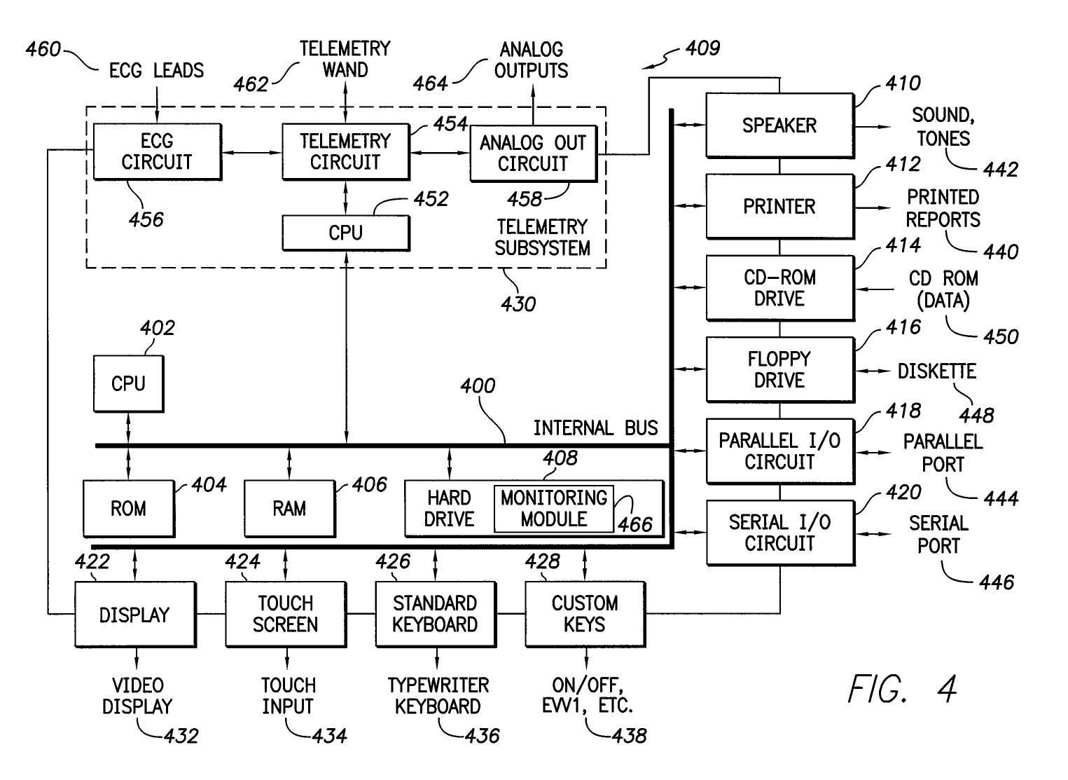

FIG. 4 illustrates a functional block diagram of an external device 409, such as a programmer, that is operated to interface with IMD 10. As described above, the external device 409 may be used by a physician or operator of the IMD 10 heart sounds and to set pacing parameters in accordance with methods described herein. The external device 108 includes an internal bus 400 that connects/interfaces with a Central Processing Unit (CPU) 402, ROM 404, RAM 406, a hard drive 408, the speaker 410, a printer 412, a CD-ROM drive 414, a floppy drive 416, a parallel I/O circuit 418, a serial I/O circuit 420, the display 422, a touch screen 424, a standard keyboard connection 426, custom keys 428, and a telemetry subsystem 430. The internal bus 400 is an address/data bus that transfers information between the various components described herein. The hard drive 408 may store operational programs as well as data, such as cardiogenic impedance parameters and the electrophysiologic response parameters. The hard drive 408 includes a monitoring module 466 that monitors the cardiogenic impedance parameters and the electrophysiologic response parameters in order to identify a potential cause of pulmonary edema.

The CPU 402 typically includes a microprocessor, a micro-controller, or equivalent control circuitry, designed specifically to control interfacing with the external device 409 and with the IMD 10. The CPU 402 may include RAM or ROM memory 404, logic and timing circuitry, state machine circuitry, and I/O circuitry to interface with the IMD 10. The display 422 (e.g., may be connected to the video display 432) and the touch screen 424, display graphic information relating to the IMD 10. The touch screen 424 accepts a user's touch input 434 when selections are made. The keyboard 426 (e.g., a typewriter keyboard 436) allows the user to enter data to the displayed fields, as well as interface with the telemetry subsystem 430. Furthermore, custom keys 428 turn on/off 438 (e.g., EVVI) the external device 409. The printer 412 prints copies of reports 440 for a physician to review or to be placed in a patient file, and speaker 410 provides an audible warning (e.g., sounds and tones 442) to the user. The parallel I/O circuit 418 interfaces with a parallel port 444. The serial I/O circuit 420 interfaces with a serial port 446. The floppy drive 416 accepts diskettes 448. Optionally, the floppy drive 416 may include a USB port or other interface capable of communicating with a USB device such as a memory stick. The CD-ROM drive 4714 accepts CD ROMs 450.

The telemetry subsystem 430 includes a central processing unit (CPU) 452 in electrical communication with a telemetry circuit 454, which communicates with both an ECG circuit 456 and an analog out circuit 458. The ECG circuit 456 is connected to ECG leads 460. The telemetry circuit 454 is connected to a telemetry wand 462. The analog out circuit 458 includes communication circuits to communicate with analog outputs 464. The external device 108 may wirelessly communicate with the IMD 10 and utilize protocols, such as Bluetooth, GSM, infrared wireless LANs, HIPERLAN, 3G, satellite, as well as circuit and packet data protocols, and the like. Alternatively, a hard-wired connection may be used to connect the external device 108 to the IMD 10.

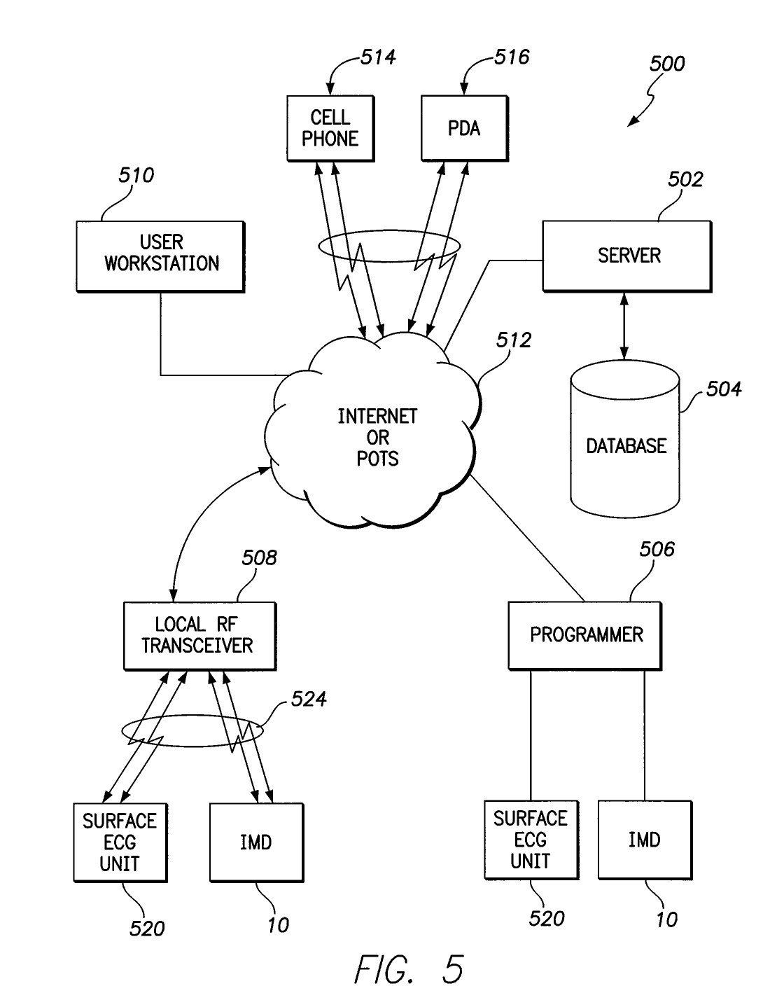

FIG. 5 illustrates a distributed processing system 500 in accordance with one embodiment. The distributed processing system 500 includes a server 502 connected to a database 504, a programmer 506 (e.g., similar to external device 108), a local RF transceiver 508 and a user workstation 510 electrically connected to a communication system 512. The communication system 512 may be the internet, a voice over IP (VoIP) gateway, a local plain old telephone service (POTS) such as a public switched telephone network (PSTN), a cellular phone based network, and the like. Alternatively, the communication system 512 may be a local area network (LAN), a campus area network (CAN), a metropolitan area network (MAN), or a wide area network (WAM). The communication system 512 serves to provide a network that facilitates the transfer/receipt of information such as cardiogenic impedance parameters and electrophysiologic response parameters.

The server 502 is a computer system that provides services to other computing systems over a computer network. The server 502 controls the communication of information such as cardiogenic impedance parameters and electrophysiologic response parameters. The server 502 interfaces with the communication system 512 to transfer information between the programmer 506, the local RF transceiver 508, the user workstation 510 as well as a cell phone 514, and a personal data assistant (PDA) 516 to the database 504 for storage/retrieval of records of information. On the other hand, the server 502 may upload raw cardiac signals from a surface ECG unit 520 or the IMD 10 via the local RF transceiver 508 or the programmer 506.

The database 504 stores information such as the measurements for the cardiogenic impedance parameters, the electrophysiologic response parameters, and the like, for a single or multiple patients. The information is downloaded into the database 504 via the server 502 or, alternatively, the information is uploaded to the server from the database 504. The programmer 506 is similar to the external device 108 and may reside in a patient's home, a hospital, or a physician's office. Programmer 506 interfaces with the surface ECG unit 520 and the IMD 10. The programmer 506 may wirelessly communicate with the IMD 10 and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 506 to the IMD 10. The programmer 506 is able to acquire cardiac signals from the surface of a person (e.g., ECGs), intra-cardiac electrogram (e.g., IEGM) signals from the IMD 10, and/or values of cardiogenic impedance parameters and electrophysiologic response parameters from the IMD 10. The programmer 506 interfaces with the communication system 512, either via the internet or via POTS, to upload the information acquired from the surface ECG unit 520 or the IMD 10 to the server 502.

The local RF transceiver 508 interfaces with the communication system 512, via a communication link 524, to upload values of physiologic indices acquired from the surface ECG unit 520 and/or cardiogenic impedance parameters and electrophysiologic response parameters acquired from the IMD 10 to the server 502. In one embodiment, the surface ECG unit 520 and the IMD 10 have a bi-directional connection with the local RF transceiver via a wireless connection. The local RF transceiver 508 is able to acquire cardiac signals from the surface of a person, intra-cardiac electrogram signals from the IMD 10, and/or the values of cardiogenic impedance parameters and electrophysiologic response parameters from the IMD 10. On the other hand, the local RF transceiver 508 may download stored cardiogenic impedance parameters, electrophysiologic response parameters, cardiac data, and the like, from the database 504 to the surface ECG unit 520 or the IMD 10.

The user workstation 510 may interface with the communication system 512 via the internet or POTS to download values of the cardiogenic impedance parameters and electrophysiologic response parameters via the server 502 from the database 504. Alternatively, the user workstation 510 may download raw data from the surface ECG unit 520 or IMD 10 via either the programmer 506 or the local RF transceiver 508. Once the user workstation 510 has downloaded the cardiogenic impedance parameters and electrophysiologic response parameters, the user workstation 510 may process the information in accordance with one or more of the operations described above in connection with the process 500 (shown in FIG. 5). The user workstation 510 may download the information and notifications to the cell phone 514, the PDA 516, the local RF transceiver 508, the programmer 506, or to the server 502 to be stored on the database 504. For example, the user workstation 510 may communicate an identified potential cause of pulmonary edema to the cell phone 514 of a patient or physician.

FIG. 6 illustrates a chart showing hypothetical examples of how heart sound measurements, over certain portions of the cardiac cycle, may relate to different values for AV delay. The horizontal axis denotes AV delay in milliseconds, while the vertical axis represents heart sound intensity over an exemplary range of intensities (e.g. 20 to 90). FIG. 6 includes two different hypothetical examples 610 and 650 of data points 612-616 and 652-656 such as for two different devices measuring a single patient, for two different patients or for two different groups of patient populations. The data points 612-616 with intensities between 30 and 50 correspond to one example, while the data points 652-656 with intensities between 51 and 80 correspond to the second example. In example 610, data points 612-616 are illustrated for different AV delays ranging between 20 msecs. and 105 msecs. In example 650, data points 652-655 are illustrated for the same AV delays between 20 msecs. and 105 msecs. As shown in FIG. 6, when the AV delay is set to a very short time period, such as 25 msecs., the heart sound intensity at data point 612 is approximately 50 in example 610, while the heart sound intensity at data point 652 in example 650 is approximately 75.

In example 650, the heart sound intensities begin around 75 (corresponding to an AV delay of 20 msecs.) but then slightly increase, approaching 80 at data points 653 and 654 (corresponding to AV delays of 40 and 65). As the AV delay increases to exceed 65 msecs., in the example A50, the heart sound intensity falls off sharply at data point 655 (to approximately 64) and even further at data point 655 (approximately 51).

In example 610, the data points 612-616 show that the heart sounds intensity begin near a maximum value with a shorter AV delay. More specifically, at data point 612, the heart sound intensity is approximately 50, and is maintained near 50 at data point 613. However, as the AV delay is extended above 60 msecs., data points 614-616 show that the heart sound intensity falls off sharply to below 40, approaching 30 at data point 616.

The foregoing examples illustrate that the heart sound intensity, at least over certain portions of the cardiac cycle, drops to a notable lower level when the AV delay is extended. While not illustrated, as the AV delay is extended even further, at some point the heart sound intensity begins to increase again. Thus, the heart sound intensity exhibits a local minimum that corresponds to a limited duration of the range for AV delay.

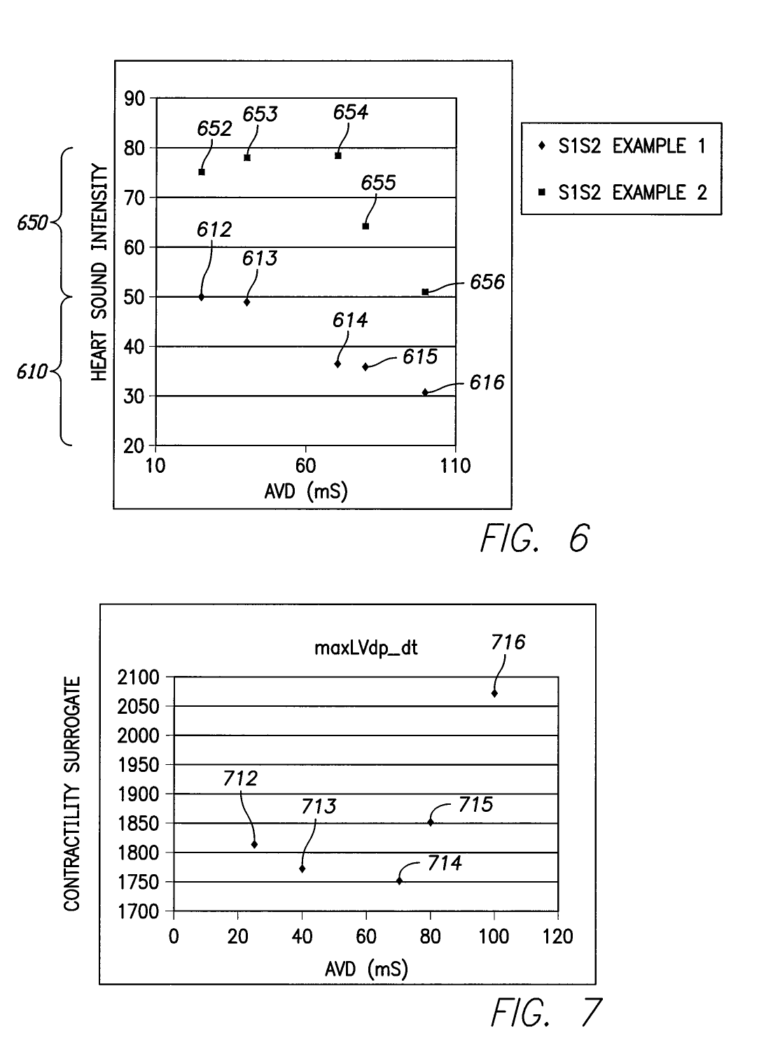

FIG. 7 illustrates a hypothetical contractility behavior plotting a potential relation between AV delay (along the horizontal axis) and maximum left ventricular pressure per unit time (maxLVdp_dt) during a cardiac cycle. In FIG. 7, a set of data points 712-716 are shown for which the AV delay is set to match the data points 612-616 in FIG. 6. The maximum change in left ventricular pressure per unit time during the cardiac cycle is believed to be a good approximation of contractility, and thus is referred to as a contractility surrogate. In the example of FIG. 7, the data point 712 illustrates that, for the hypothetical patient or patient population, when the AV delay is set slightly greater than 20 msecs., the maximum LVdp/dt was approximately 1800. When the AV delay was extended to 40 msecs. and then 70 msecs. (corresponding to data points 713 and 714), the maximum LVdp/dt dropped to 1755 and 1750, respectively. As the AV delay is further extended to 80 and then 100 msecs. (corresponding to data points 715 and 716), the maximum LVdp/dt increases to 1850 and then above 2050, respectively. The data points in FIG. 7 illustrate that an AV delay of approximately 100 msecs. achieves large contractility, through the surrogate maximum LVdp/dt. Increased contractility yields an improvement in overall hemodynamic performance of the heart. While not shown, as the AV delay is further extended, at some greater length, the maximum LVdp/dt begins to drop again and continues to fall. Hence, a local maximum in LVdp/dt is exhibited at or around 100 to 120 msec.

As explained throughout, methods and systems are provided herein to determine pacing parameters, such as the AV delay that, if implemented, would potentially yield improved hemodynamic performance through an increased contractility surrogate (as measured in one way through the maximum LVdp/dt).

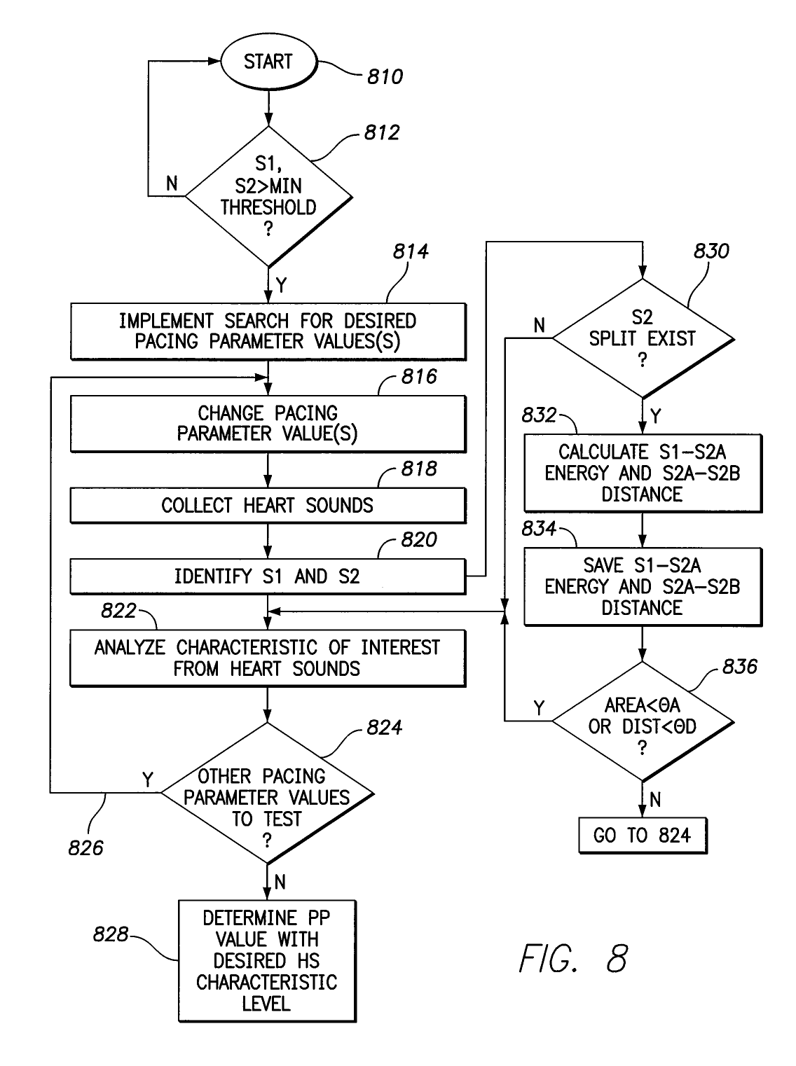

FIG. 8 illustrates one method for determining a value of at least one pacing parameter for an IMD 10 in accordance with an embodiment. Beginning at 810, the process starts by obtaining heart sound signals, from a sound sensor, such as an accelerometer and the like. At 812, the process determines whether the heart sounds that occur in the detection windows for S1 and S2 exceed a predetermined minimum threshold. By way of example, the S1 and S2 minimum thresholds may be programmed at the time of manufacture, or by the clinician during or after implantation through an external programmer. The threshold test at 812 is performed to determine whether the heart sound signals are sufficiently strong to perform the subsequent determination for pacing parameters. When the heart sounds at 812 do not exceed the minimum threshold, flow returns to 810 and the process is repeated until strong enough heart sounds are collected. Optionally, the determination at 812, may be omitted entirely and the subsequent process of 814 to 828 may be performed without concern for whether the S1 and S2 heart sounds exceed any minimum threshold.

At 814, the process begins implementation of a search for desired pacing parameter values. For example, at 814, initial values for certain pacing parameters may be obtained from memory in the IMD memory in a programmer, or based on recently collected physiologic information, such as cardiac signals, pressure measurements within one or more chambers of the heart, impedance measurements, sound measurements and the like. At 816, the current pacing value(s) are changed. For example, the pacing parameter may correspond to the AV delay, the VV delay, the VA delay, atrial and ventricular electrode combinations for pacing, atrial and ventricular electrodes to use for sensing, timing delays between left ventricular electrodes, time delays between left atrial electrodes, timing delays between LA and LV electrodes, and the like. Optionally, the pacing parameter may designate the pacing mode, such as the chambers of the heart where sensing occurs, the chambers of the heart where pacing occurs, which LV electrodes to use and the like. As a further option, the pacing parameter may represent the combination of electrodes used to deliver pacing pulses. The pacing parameter(s) may include one or more combinations of the above listed examples, as well as other parameters.

At 816, the value for one or more of the pacing parameters is changed. It is recognized that, during a first iteration through FIG. 8, the pacing parameter may not be changed at 816. Optionally, the pacing parameter value(s) may be changed by a predetermined programmed increment (e.g., increased or decreased by a set amount). The amount or nature of change in the value for the pacing parameter may be automatically determined by the IMD or external programmer. Optionally, the amount of change in the pacing parameter may be a percentage of the current value, where the percentage is derived from physiologic signals (e.g., cardiac signals, impedance signals, pressure signals and the like). As one example, the pacing parameter may represent the AV delay with an initial AV delay set to 20 msecs., at 814. At 816, the AV delay may be changed by increasing the AV delay in 5 millisecond steps every iteration through 816. Optionally, the change may represent a series of steps through different combinations of LV electrodes, or a series of incremental changes in the pacing delay between LV electrodes or the delay between pacing in the RV and select LV electrodes.

At 818, a new series of heart sounds are collected for one or more cardiac cycles, while the IMD 10 operates using the pacing parameter value set in 816. In the foregoing example, when the AV delay is increased to 25 msecs., at 818, heart sounds, may be collected over 1 to 10 or more cardiac cycles, while the IMD 10 operates with the AV delay of 25 msecs. When heart sounds for multiple cardiac cycles are collected, each cardiac cycle may be separated (e.g., based on a marker such as the R-wave). The heart sounds for each cardiac cycle may be processed separately at 820 and 822. Alternatively, the heart sounds for each cardiac cycle may be aligned with one another and summed to form a composite signal for heart sounds. For example, an ensemble of heart sounds for 3, 5 or 10 cardiac cycles may be temporally aligned based on a marker such as the peak of the R-wave and summed. Optionally, the ensemble of heart sounds may be aligned through auto correlation, cross-correlation, or other techniques and then summed.

At 820, the S1 and S2 heart sounds are identified from the collected heart sounds. To identify the S1 and S2 heart sounds, the process may first establish S1 and S2 detection windows that are overlaid upon the heart sounds. The S1 and S2 detection windows are positioned to start a predetermined offset time after a marker of interest. For example, the S1 and S2 detection windows may be offset to start 100 msec. and 250 msec., respectively, after the peak R-wave. For example, the identification at 820 may include an identification of the peak in the S1 heart sound and the peak in the S2 heart sound during corresponding detection windows. Optionally, the identification may determine i) the center of the S1 and S2 heart sounds, ii) the durations of the S1 and S2 heart sounds, and iii) the peak amplitude of the S1 and S2 heart sounds. When identifying the center of the S1 and S2 heart sounds, the center may represent the temporal center or the center of the energy content for the S1 heart sound and the temporal center or the center for the energy content for the S2 heart sound.

When heart sounds for multiple cardiac cycles are collected at 818, each cardiac cycle may be processed individually at 820 and 822. Alternatively, when heart sounds for an ensemble of cardiac signals are combined, the composite heart sounds may be processed at 820 to identify a single composite S1 heart sound and a single composite S2 heart sound. The composite heart sounds are then analyzed at 822.

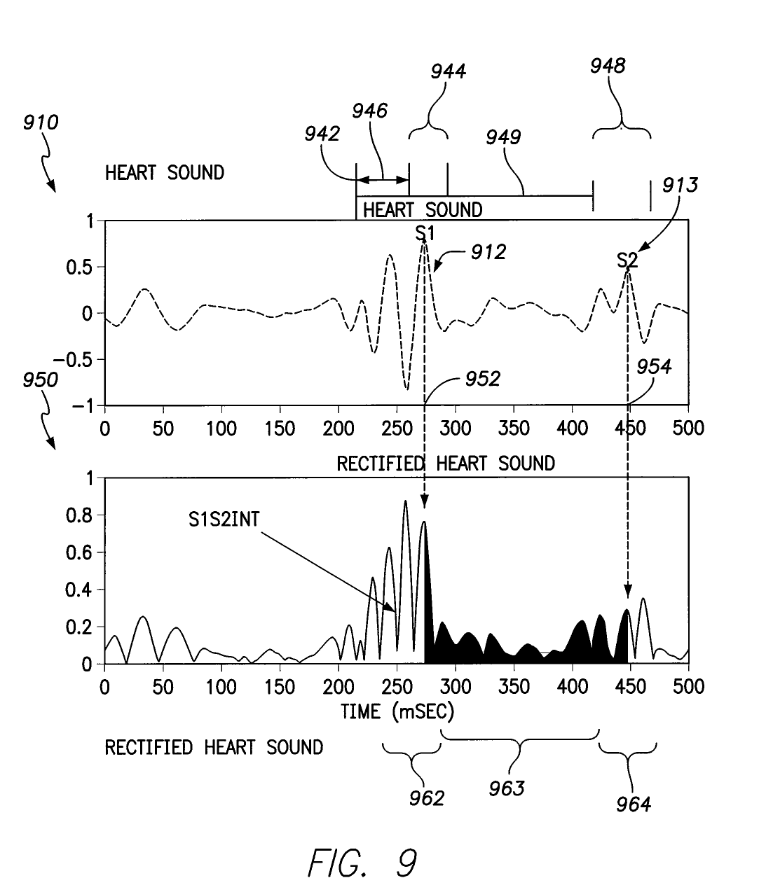

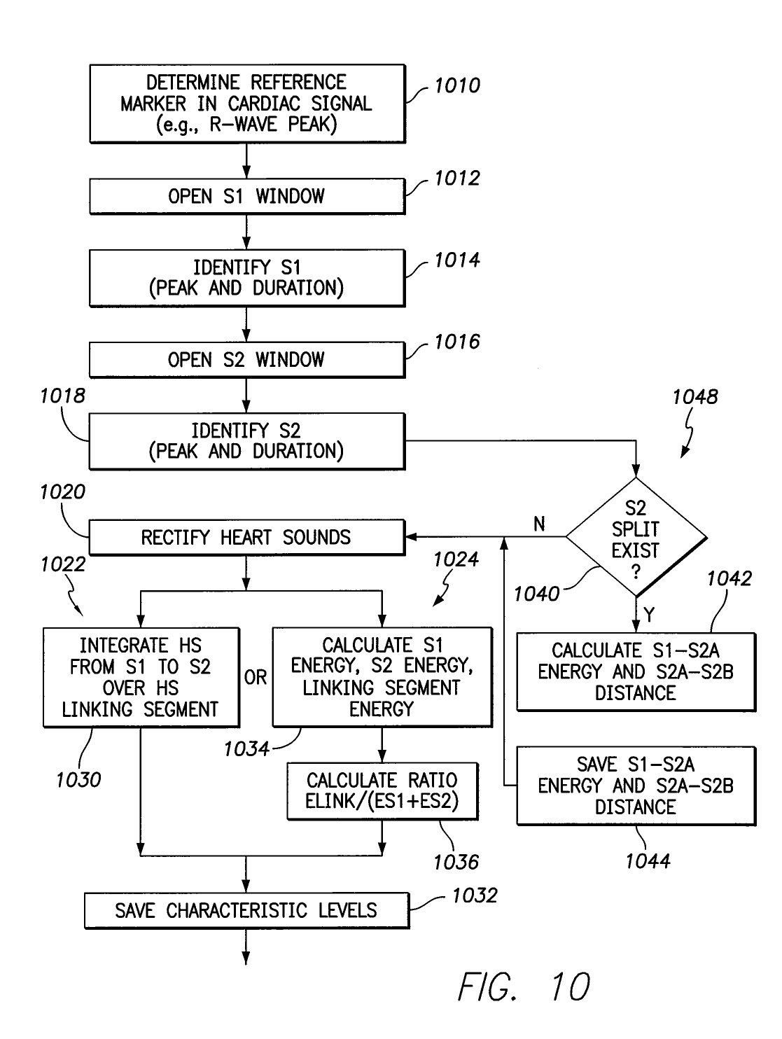

Next, the operations at 818 and 820 will be described in connection with FIG. 9 for a single cardiac cycle, but it is understood that the same process may be implemented for sets of cardiac signals or ensembles of cardiac signals.

FIG. 9 illustrates exemplary heart sounds collected over a single cardiac cycle or composite heart sounds combined from an ensemble of cardiac cycles. The upper graph 910 plots an exemplary signal for heart sounds spanning a time period of 500 msecs. (plotted along the horizontal axis). The vertical axis plots a signal measured at a sound sensor. In the example of FIG. 9, the sound sensor represents an accelerometer and a voltage or current signal produced therefrom oscillate between positive and negative levels between a normalized range of 1 to −1. The region generally denoted at 912 represents the S1 heart sound segment, while the region generally denoted at 913 represents the S2 heart sound segment.

As one example, the process may analyze cardiac signals to identify a marker, such as the peak of the R-wave (denoted at 942). The process may then set an S1 detection window 944 to begin a programmed period of time after the marker 942. This programmed delay time 946 may be a set number of milliseconds or a percentage of the cardiac cycle length and the like. The length of the S1 detection window 944 may also be programmed. The process may similarly set an S2 detection window 948, beginning a delay time 949 after the marker 942. The process only searches for S1 and S2 peaks during the corresponding S1 and S2 detection windows 944 and 948.

During the identification operation at 820 (FIG. 8), the S1 heart sounds and S2 heart sounds are identified. For example, the identification at 820 may determine the time denoted at 952 as the S1 heart sound and the time denoted at 954 as the S2 heart sound. By way of example, the S1 and S2 heart sound may be identified as the peak positive levels in the heart sounds within the detection windows 944 and 948.

The heart sounds may be collected over multiple cardiac cycles and separately analyzed to identify multiple S1 and S2 heart sounds at 820, or combined and analyzed to identify composite S1 and S2 heart sounds.

Returning to FIG. 8, next at 830, the process determines whether there is a split in the heart sounds. The operations at 830 to 836 may be omitted and flow may move from 820 to 822. The operations at 830-836 seek to identify splits in the S2 heart sound. During inspiration, the chest wall expands and causes the intra-thoracic pressure to become more negative which allows the lungs to fill with air and expand. While doing so, it also induces an increase in venous blood return from the body into the right atrium via the superior and inferior vena cavae, and into the right ventricle by increasing the pressure gradient. Simultaneously, there is a reduction in blood volume returning from the lungs into the left ventricle. Since there is an increase in blood volume in the right ventricle, the pulmonary valve (P2 component of S2) stays open longer during ventricular systole due to an increase in ventricular emptying time, whereas the aortic valve (A2 component of S2) closes slightly earlier due to a reduction in left ventricular volume and ventricular emptying time. Thus the P2 component of S2 is delayed relative to that of the A2 component. This delay in P2 versus A2 is heard as a slight broadening or even “splitting” of the second heart sound. During expiration, the chest wall collapses and decreases the negative intrathoracic pressure (compared to inspiration). Therefore, there is no longer an increase in blood return to the right ventricle versus the left ventricle and the right ventricle volume is no longer increased. This allows the pulmonary valve to close earlier such that it overlaps the closing of the aortic valve, and the split is no longer heard.

It may be physiologically normal to hear a slight “splitting” of the second heart tone. However, different types of split S2 can be associated with medical conditions. For example, while a split during inspiration may be normal, a split during expiration may indicate pathology (e.g., Aortic stenosis, hypertrophic cardiomyopathy, left bundle branch block). When splitting does not vary with inspiration, it may be termed a “fixed split S2” and may be due to a septal defect, such as an atrial septal defect (ASD) or ventricular septal defect (VSD). The ASD or VSD creates a left to right shunt that increases the blood flow to the right side of the heart, thereby causing the pulmonary valve to close later than the aortic valve independent of inspiration/expiration.

A bundle branch block either LBB or RBB, (although RBB is known to be associated only with S1 split), may produce continuous splitting but the degree of splitting will still vary with respiration. When the pulmonary valve closes before the aortic valve, this is known as a “paradoxically split S2”.

In accordance with certain embodiments herein, the heart sounds are analyzed to identify a split S2 heart sound and to identify pacing parameters that reduce or minimize the degree/amount of S2 split. At 830, by way of example, a split in the heart sound S2 may be determined by analyzing the S2 heart sound for more than one peak. For example, the S2 heart sound may be compared to an S2 threshold, and a split may be declared when the S2 heart sound exceeds the S2 threshold in two distinct regions separated by a time delay. The time delay between the S2 heart sound regions may be predetermined, programmed and/or dynamically updated based on real time physiologic measurements. Optionally, the S2 heart sound may be analyzed to determine whether two or more absolute peaks exist and then the process may determine the spacing between these absolute peaks. Alternatively, when a first S2 (S2A) heart sound peak is identified, a split detection window may be overlaid on the remaining portion of the S2 heart sound. If a 2^{nd} peak (S2B) occurs within the split detection window, then a split S2 heart sound is declared. The split detection window may start a predetermined time after the first S2 heart sound and then end before another type of heart sound may occur.

Alternatively, the S2 heart sound may be passed through a low pass filter to form a smoothed, filtered heart sound. The smoothed, filtered heart sound signal is then analyzed to determine changes in the slope of the heart sound signal. The points in time at which the slope changes from positive to negative may be used to identify local peaks. These local peaks (S2A and S2B) are compared to determine a time spacing there between. When the S2 heart sound signal exhibits two peaks that are separated in time by sufficient time spacing, this signal is declared to be split into S2A and S2B heart sounds at 830 and flow moves to 832.

At 832, the process determines the area (e.g., the energy) under the heart sound signal between S1 and the first S2 heart sound (S1-S2A area or S1-S2A energy). At 832, the process also identifies the distance between the first and second S2 heart sounds (S2A-S2B distance). This distance may be between the peaks of S2A and S2B. Optionally, the S2A-S2B distance may be between the centers of the S2A and S2B heart sounds. When identifying the center of the S2A and S2B heart sounds, the center may represent the temporal center or the center of the energy content for the S2A heart sound and the temporal center or the center for the energy content for the S2B heart sound. Optionally, the area or energy between S1 and S2B may be determined.

At 834, the process saves one or more of the S1-S2A energy under the heart sound signal between S1 and S2A, the S1-S2B energy under the heart sound signal between S1 and S2B and the distance between S2A and S2B as potential characteristics of interest.

At 836, the process compares one or more of the S1-S2A energy with a predetermined area or energy threshold GA, the S1-S2B energy with a predetermined area or energy threshold GB, and compares the S2A-S2B distance with a predetermined distance threshold OD. If the S1-S2A energy, S1-S2B energy and/or the S2A-S2B distance exceed the corresponding threshold ΘA, ΘB and/or ΘD, then the S2 split heart sounds are declared to not be suitable to base changes in pacing parameters thereon. Hence, flow moves to 824 and the process continues.

When at 836, the S1-S2A energy, S1-S2B energy and/or the S2A-S2B distance are determined to fall within and not exceed the corresponding threshold ΘA, ΘB and/or ΘD, then the split S2 heart sounds are declared to be suitable for further analysis and to be used as the basis to change pacing parameters. Hence, flow continues to 822.

At 822, one or more predetermined characteristics of interest for the heart sounds are analyzed. The heart sounds of interest have S1, S2 and linking segments. The heart sounds of interest may also include a split S2 and thus have an S2A portion, an S2B portion and a split segment between S2A and S2B. The S1 segment is associated with initial systole activity. The S2 segment is associated with initial diastole activity. The linking segment is associated with at least a portion of heart activity occurring between the S1 and S2 segments during a systolic interval between the initial systole and diastole activity. At 822, the characteristic of interest is representative of a degree of blood turbulence during the systolic interval which corresponds to the ventricular ejection phase of the cardiac cycle. The characteristic of interest is not simply limited to the S1 heart sound and not simply limited to the S2 heart sound. Instead, the characteristic of interest may solely relate to a phase of the cardiac cycle beginning after S1 and ending before S2. Alternatively, the characteristic of interest may represent a relation between S1, S2 and the phase therebetween. The characteristic of interest for the heart sounds may represent the intensity of the heart sounds, the energy content of the heart sounds, ratios between the energy content within different segments of the heart sounds, and the like.

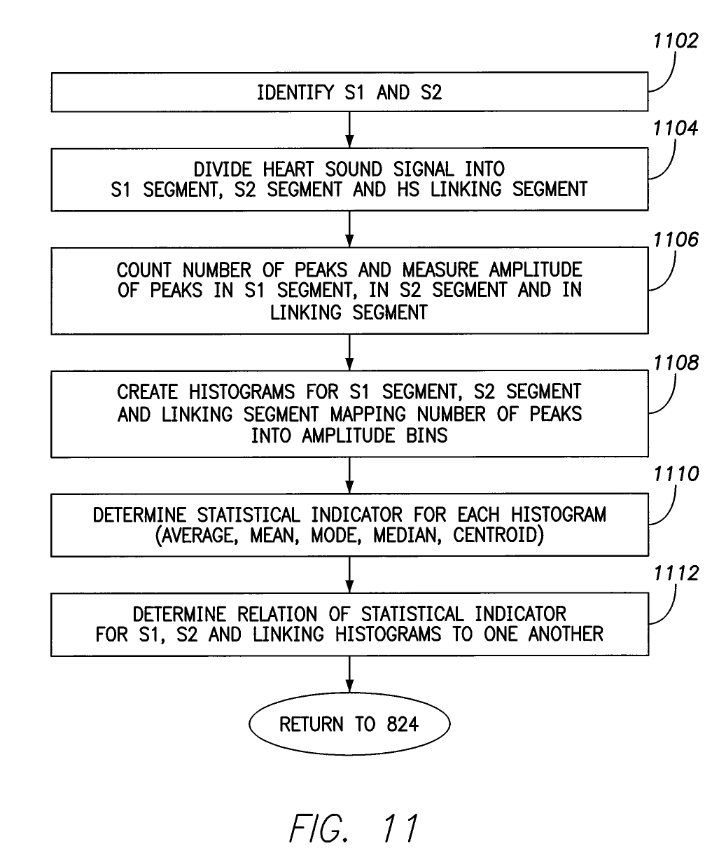

The characteristic of interest may relate to a phase of a split S2 heart sound beginning at S2A and ending at S2B. Alternatively, the characteristic of interest may represent a relation between the split sounds, S2A and S2B, and the phase therebetween. The characteristic of interest for the split S2A, S2B heart sounds may represent the intensity of the S2A, S2B heart sounds, the energy content of the S2A, S2B heart sounds, ratios between the energy content within different segments of the S2A, S2B heart sounds, and the like. The operations at 820 and 822 are discussed hereafter in more detail in connection with FIG. 10. It should be realized that to the extent a common operation is shown in FIG. 8 and in FIG. 10, the operation may not be repeated in FIG. 10 for the same cardiac cycle. Instead, the results obtained in FIG. 8 may simply be used. For example, the operation at 1042 in FIG. 10 may not be repeated if the operations at 832 in FIG. 8 have already calculated the S1-S2A energy and S2A-S2B distance for a current cardiac cycle. The operations 1012-1018 in FIG. 10 may not be repeated if the operations at 820 have already calculated the S1 and S2 peak and duration.

FIG. 10 illustrates a processing sequence for identifying S1 and S2 heart sounds and/or split heart sounds S2A, S2B for a single cardiac cycle or ensemble of cardiac cycles and analyzing the heart sounds for a characteristic of interest. Beginning at 1010, the process identifies a reference marker in a cardiac signal, such as the peak of the P-wave, R-wave, etc. At 1012, an S1 detection window is opened, a programmed delay time following the reference marker. At 1014, the S1 heart sound is identified, such as by identifying the peak in the heart sounds during the S1 detection window 944 (FIG. 9). Next, at 1016, an S2 detection window is opened. At 1018, the S2 heart sound is identified, such as by identifying the peak in the heart sounds that occur during the S2 detection window 948 (FIG. 9).

At 1014 and 1018, one or more features of S1 and S2 may be identified. For example, peak and duration for S1 and S2 may be identified, including start and end times for each of the S1 and S2 segments.

At 1040, the process determines whether there is a split in the heart sounds. By way of example, a split in the heart sound S2 may be determined by analyzing the S2 heart sound for more than one peak or in various other manners described herein and apparent here from. When the S2 heart sound signal exhibits two peaks that are separated in time by sufficient time spacing, this signal is declared to be split into S2A and S2B heart sounds at 1040 and flow moves to 1042.

At 1042, the process determines the area under the heart sound signal between S1 and the first S2 heart sound (S1-S2A energy). At 1042, the process also identifies the distance between the first and second S2 heart sounds (S2A and S2B). This distance may be between the peaks of S2A and S2B. Optionally, the distance may be between the centers of the S2A and S2B heart sounds. When identifying the center of the S2A and S2B heart sounds, the center may represent the temporal center or the center of the energy content for the S2A heart sound and the temporal center or the center for the energy content for the S2B heart sound.

At 1044, the process saves one or more of the S1-S2A energy under the heart sound signal between S1 and S2A, the S1-S2B energy under the heart sound signal between S1 and S2B and the distance between S2A and S2B as characteristics of interest. Next flow moves to 1020. Optionally, 1040 to 1044 may be skipped or omitted if the same test and the same information is tested, calculated and saved at 830-834 in FIG. 8.

At 1020, the heart sounds are rectified to form a positive signal within a normalized range of 0 to 1. Next, one or more of multiple processes may be followed to analyze one or more desired characteristics of the heart sounds. In FIG. 9, a lower graph 950 in which a series of bracket are illustrated as examples of the different S1, S2 and linking segments 962-964 into which the heart sound signals may be divided. The S1 segment 962 denotes a region generally attributed to heart sound S1, while S2 segment 964 is attributed to heart sound S2. The relatively long intervening period, refers to a heart sound linking segment 963 that is not directly attributed to S1 or S2, yet during which heart sounds are produced. In at least certain embodiments described herein, it may be desirable to set pacing parameters to values that correspond to reduced or limited intensity or energy content within the linking segment 963. By reducing or limiting the intensity or energy content in linking segment 963, pacing parameters may be set to values that achieve increased contractility and improved hemodynamic performance by the heart.

In the example of FIG. 10, branches 1022, 1048 and 1024 are illustrated to denote parallel, serial or alternative characteristics of interest that may be analyzed. When flow moves along branch 1022, at 1030, the heart sounds are integrated for the rectified signal beginning at the S1 peak and continuing to the S2 peak over the intermediate heart sound (HS) linking segment. At 1030, the heart sounds are integrated over the entire range spanning from the S1 peak to the S2 peak including the linking segment. This integration value is then saved as a characteristic value at 1032 in one to one relation with the current pacing parameter value.

Alternatively or in addition, the heart sounds may be analyzed along branch 1024 by looking at an alternative characteristic of interest. Along branch 1024, the process first calculates the S1 energy content, S2 energy content and linking segment energy content. The process may identify the S1, S2 and linking segment energy contents by separately integrating the heart sound signals (rectified) over the corresponding S1, S2 and linking segments of the heart sounds. For example, the S1 energy content may be derived by integrating the heart sound signals within the range corresponding to S1 segment 962 (FIG. 9). The S2 energy may be derived by integrating the heart sound signals (rectified) over the range corresponding to S2 segment 964. Similarly, the linking segment energy may be derived by integrating the heart sound signals over the range corresponding to linking segment 963.

At 1036, one or more different types of relations may be calculated between the S1, S2 and linking segment energies. For example, a ratio (Elink/Es1+Es2) may be calculated between the amount of energy in the linking segment 963 relative to the sum of the amount of energy in the S1 and S2 segments 962 and 964. As the heart sound signal increases during the linking segment 963, the ratio increases. It may be desirable to adjust the pacing parameters until the energy obtained during the linking segment 963 approaches a relatively low level or reaches a minimum. Once the ratio is calculated 1036, this ratio is saved as a characteristic value at 1032 in a one to one relation with the current pacing parameter value(s).

When flow moves along the branch 1048, the split S2 heart sounds are integrated over the range spanning from the S1 peak to the S2A peak. This split S2 integration value, and the distance between S2A and S2B are then saved as characteristic values at 1032 in one to one relation with the current pacing parameter values. It may be desirable to adjust the pacing parameters until the energy obtained during the S1-S2A energy, the energy obtained during the S1-S2B energy and/or the duration of the S2A-S2B time delay approach relatively low levels or reaches a minimum. As the operations discussed herein are repeated, the process seeks to reduce the S2 split by selecting pacing parameters that are associated with i) a low or minimum area under the curve between S1 and S2A, ii) a low or minimum area under the curve between S1 and S2B, and/or iii) a short time delay between S2A and S2B.

Returning to FIG. 8, once the heart sounds have been analyzed for the desired characteristic or characteristics of interest, flow moves to 824 where it is determined whether the pacing parameter should be adjusted to another level and retested. If yes, flow returns along 826 to 816. The operations at 816-822 and 830-836 are then repeated multiple times to build a data set of characteristic level and related pacing parameter values. Alternatively, when at 824, it is determined that all of the desired values for the pacing parameter(s) or have been tested, flow moves to 828. At 828, the stored heart sound characteristic levels are reviewed to determine the desired characteristic level. For example, the desired characteristic level may represent a minimum or a maximum depending upon which type of characteristic is analyzed. In the example of FIG. 10, it may be desirable to choose the minimum characteristic level determined at 1030, 1042 and/or 1036, thereby minimizing the amount of energy or intensity of the heart sounds during the linking segment 963 (FIG. 9) and/or to minimize the energy or duration of the split S2 heart sound. Once the desired characteristic level is identified, the corresponding pacing parameter value is matched thereto and used as a new setting for the pacing parameters.

Optionally, the characteristics of interest determined along branches 1022, 1024 and 1048 may be combined, such as through a weighted sum, to utilize all three types of characteristic information when selecting the desired pacing parameters. Alternatively, the characteristics determined along branches 1022, 1024 and 1048 may be used to separately identify three candidate pacing parameters, which are then merged to form a desired pacing parameter.

FIG. 11 illustrates an alternative process to analyze a characteristic of interest from the heart sounds. At 1102, the process identifies S1 and S2 heart sounds (similar to the operations discussed herein in connection with other embodiments). At 1104, the heart sounds, over a cardiac cycle, are divided into segments corresponding to an S1 segment, S2 segment and a linking segment (962-964 in FIG. 9). The systolic interval includes at least the linking segment, and may also include the S1 segment. At 1106, the rectified heart sounds within the S1, S2 and linking segments are separately analyzed to identify each peak therein. The peaks are counted and the amplitude of the peaks are measured.

At 1108, a set of histograms are created, with an S1 histogram for the S1 segment, an S2 histogram counting peaks that occurred during the S2 segment, and a linking histogram counting peaks that occurred during the linking segment. The histograms include contiguous non-overlapping bins for different ranges of heart sound amplitudes. The S1 histogram stores a count in each bin for the number of peaks that occurred during the S1 segment having an amplitude within the corresponding range. The S2 histogram stores a count in each bin for the number of peaks that occurred during the S2 segment having an amplitude within the corresponding range. The linking histogram stores a count in each bin for the number of peaks that occurred during the linking segment having an amplitude within the corresponding range.

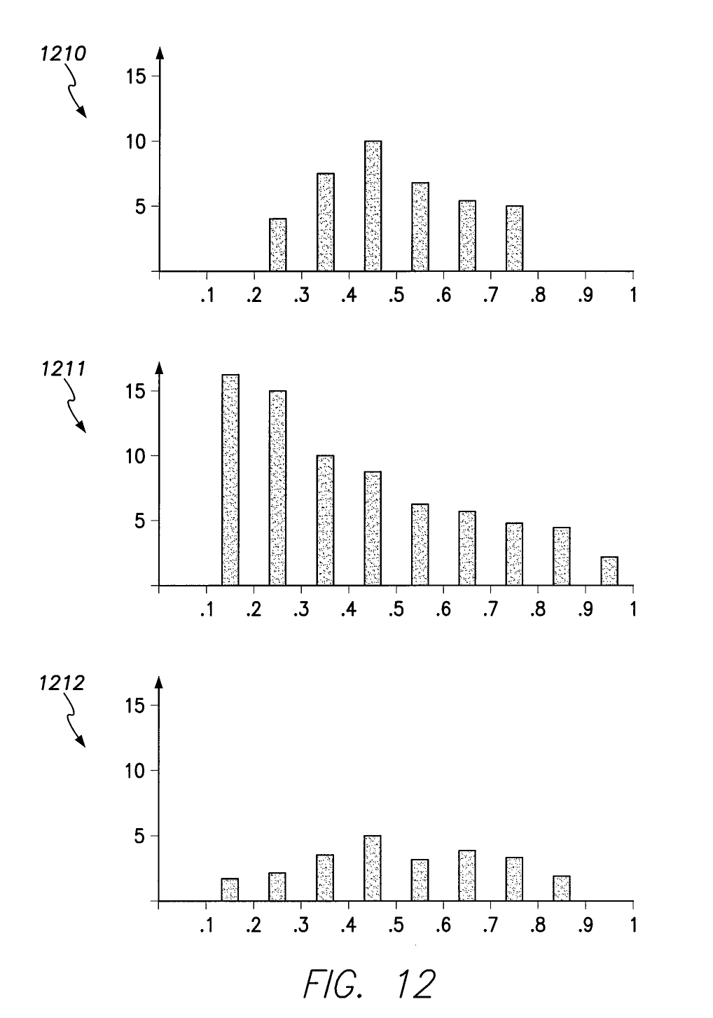

FIG. 12 illustrates a set of histograms 1210-1212 that may be created from one cardiac cycle or from an ensemble of cardiac cycles. The histograms 1210-1212 correspond to the S1, S2 and linking segments, respectively. The histograms 1210-1212 are divided into amplitude bins designated along the horizontal axis. Each histogram 1210-1212 stores a running count of the number of peaks within each amplitude bin for each of the S1, S2 and linking segments. For example, histogram 1210 illustrates that, for the S1 segment, the process counted 10 peaks in the amplitude range of 0.4 to 0.5, and only counted 3 and 5 peaks in the amplitude ranges of 0.2 to 0.3, and 0.7 to 0.8, respectively. For example, histogram 1211 illustrates that, for the linking segment, the process counted 3 peaks in the amplitude range of 0.4 to 0.5, and counted 15 and 5 peaks in the amplitude ranges of 0.2 to 0.3, and 0.7 to 0.8, respectively. For example, histogram 1212 illustrates that, for the S2 segment, the process counted 5 peaks in the amplitude range of 0.4 to 0.5, and only counted 2 and 3 peaks in the amplitude ranges of 0.2 to 0.3, and 0.7 to 0.8, respectively. The process may save separate histograms for each cardiac cycle, or alternatively, update each of histograms 1210-1212 with counts from multiple cardiac cycles (e.g. over a 30 second, 1 minute, 5 minute period).

Returning to FIG. 11, at 1110, each histogram is separately analyzed to obtain a statistical indicator representative thereof. For example, the statistical indicator may represent a moment average, mean, median, mode, centroid (center of mass), standard deviation, a Gaussian curve fit, and the like. A statistical indicator is derived for each of the S1, S2 and linking histograms.

At 1112, the statistical indicators are compared to one another to obtain one or more relations between the S1, S2, and linking histograms. These relations represent a characteristic value that is saved in connection with the current pacing parameter values. For example, the moment for the linking histogram may be compared to the moment for the S1 and/or S2 histograms. Optionally, the centroid for the linking histogram may be compared to an average of the centroids for the S1 and S2 histograms. Alternatively, a difference between the average for the S1 histogram and the average for the linking histogram may be compared to a difference between the average for the S2 histogram and the average for the linking histogram. Once the desired relation is determined, it is saved as a current characteristic value with the corresponding pacing parameter value, and flow returns to 824 (FIG. 8) to determine whether another iteration through the process of FIG. 8 is warranted.

The foregoing process, of FIG. 11, is repeated for all desired pacing parameter values. Then flow returns to 828 (FIG. 8) where a desired pacing parameter value is chosen to correspond with minimum amount of heart sound in the linking segment.

At 828, for example, an AV delay of approximately 100 msec. may have been set if the patient exhibited the behavior shown in FIGS. 6 and 7. In some examples, the pacing parameter value may be chosen that corresponds to the lowest intensity in heart sounds for the linking segment. Alternatively, the pacing parameter value may be chosen to correspond to the ratio of S1, S2 and linking segment energy content that falls below a predetermined threshold. Optionally, when a split S2 exists, the pacing parameter values may be chosen that correspond to a smallest or reduced area/energy between S1 and S2A. Alternatively, when a split S2 exists, the pacing parameter values may be chosen that correspond to a smallest or reduced time delay between S2A and S2B. Other criteria may be used to select the preferred pacing parameter value that is believed to yield a desired contractility and hemodynamic performance.

In accordance with an embodiment, an IMD is provided that uses a device-based accelerometer to measure heart sound intensity to assess the degree of blood turbulence during systolic (ejection) as well as diastolic (filling) time for a set of cardiac device therapy parameters. A desired parameter value may be chosen based on minimal heart sound intensity between S1 and S2. When the IMD represents a rate-responsive IMD, the collecting, changing and identifying operations may be repeated periodically by the rate-responsive IMD to provide real-time updates to the pacing parameter throughout operation

Further, embodiments can be implemented to apply in rate adaptive pacing where AV delay adaptation is desired. On-the-fly AV delay adaptation is possible to achieve relatively low systolic turbulence.

In accordance with an embodiment, a PSA/Programmer based system is provided with a wand having mean of acoustic sensing (microphone or accelerometer) and that will determine optimal parameters during CRT device follow-ups or at the time of implant.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A method to determine pacing parameters for an implantable medical device (IMD), the method comprising:
collecting heart sounds during the cardiac cycles, the heart sounds including sounds representative of a degree of blood flow turbulence, the heart sounds including S1 and S2 heart sounds and linking segments, the S1 segment associated with initial systole activity, the S2 segment associated with initial diastole activity, the linking segment associated with heart activity occurring during a systolic interval between the initial systole and diastole activity;
changing a value for a pacing parameter between the cardiac cycles;
analyzing a characteristic of interest from the heart sounds within at least one a) of a portion of the linking segment and b) of a split S2 heart sound, wherein the characteristic of interest is indicative of at least one of i) an amount of heart sounds over at least a portion of the systolic interval between the initial systole and diastole activity and ii) an amount of split in the S2 heart sound, the level of the characteristic of interest changing as the pacing parameter is changed; and
setting a desired value for the pacing parameter based on the characteristic of interest from the heart sounds from the linking segment.

2. The method of claim 1, wherein the analyzing operation includes identifying S1 and S2 peaks associated with the initial systole and diastole activity, respectively, and integrating the heart sounds over the time period between the S1 and S2 peaks.

3. The method of claim 1, wherein the analyzing operation determines an energy content within the linking segment, the energy content within the linking segment excluding an energy content within the S1 and S2 segments, the setting operation reducing the energy content within the linking segment to below a predetermined level.

4. The method of claim 1, wherein the analyzing operation determines S1 energy content associated with the S1 segment, S2 energy content associated with the S2 segment, and linking energy content associated with the linking segment, the S1, S2 and linking energy contents being mutually exclusive of one another, the setting operation limiting a ratio of the S1, S2 and linking energy contents to a predetermined level.

5. The method of claim 1, wherein the characteristic analyzed during the analyzing operation identifies at least one of intensity or energy content of the heart sounds as the amount over an entirety of the systolic interval following the S1 heart sound.

6. The method of claim 1, further comprising determining a minimum level for the heart sounds from a collection of the heart sounds collected over multiple cardiac cycles, the setting operation setting the desired value to correspond to the minimum level for the heart sounds.

7. The method of claim 1, wherein the collecting operation is performed during implantation of the IMD, the collecting operation utilizes an external programmer to control the collecting, changing and analyzing operations.

8. The method of claim 1, wherein the collecting operation includes deriving heart sounds from signals produced by an accelerometer within the IMD.

9. The method of claim 1, wherein the analyzing operation includes analyzing an energy or time delay in the split S2 heart sound as the characteristic of interest, the energy or time delay in the split S2 heart sound changing as the pacing parameter is changed; and wherein the setting operation includes setting a desired value for the pacing parameter based on at least one of the energy or time delay in the split S2 heart sound.

10. The method of claim 1, wherein the pacing parameter represents at least one of an AV delay, a W delay and a VA delay, and the changing operation changes at least one of the AV delay, the VV delay and VA delay in order reduce systolic turbulence and regurgitation.

11. A system, comprising:
one or more inputs configured to be coupled to at least one lead having electrodes to sense intrinsic events and to deliver pacing pulses over cardiac cycles;
a sensor for collecting heart sounds during cardiac cycles, the heart sounds including sounds representative of a degree of blood flow turbulence, the sensor collecting the heart sounds that include S1 and S2 heart sounds and linking segments, the S1 segment associated with initial systole activity, the S2 segment associated with initial diastole activity, the linking segment associated with heart activity occurring during a systolic interval between the initial systole and diastole activity;
a controller to control delivery of pacing pulses based on pacing parameters, the controller to change a value for at least one of the pacing parameters between the cardiac cycles;
an analysis module to analyze a characteristic of interest from the heart sounds within at least one of a) a portion of the linking segment and b) a split S2 heart sound, wherein the characteristic of interest is indicative of at least one of i) an amount of the heart sounds over at least a portion of the systolic interval between the initial systole and diastole activity and ii) an amount of split in the S2 heart sound, the level of the characteristic of interest changing as the pacing parameter is changed; and
a setting module to set a desired value for the pacing parameter based on the characteristic of interest from the heart sounds from the linking segment.

12. The system of claim 11, wherein the analysis module identifies S1 and S2 peaks associated with the initial systole and diastole activity, respectively, and integrates the heart sounds over the time period between the S1 and S2 peaks.

13. The system of claim 11, wherein the analysis module determines an energy content within the linking segment, the energy content within the linking segment excluding an energy content within the S1 and S2 segments, the setting operation reducing the energy content within the linking segment to below a predetermined level.

14. The system of claim 11, wherein the analysis module determines S1, S2 and linking energy contents individually associated with the S1, S2 and linking segments, respectively, the S1, S2 and linking energy contents being mutually exclusive of one another, the setting module limiting a ratio of the S1, S2 and linking energy contents to a predetermined level.

15. The system of claim 11, wherein the characteristic analyzed by the analysis module identifies at least one of intensity or energy content as the amount of the heart sounds over an entirety of the systolic interval following the S1 heart sound.

16. The system of claim 11, wherein the analysis module determines a minimum level for the heart sounds from a collection of the heart sounds collected over multiple cardiac cycles, the setting module setting the desired value to correspond to the minimum level for the heart sounds.

17. The system of claim 11, further comprising an external programmer coupled to the sensor for collecting the heart sounds during implantation of the IMD, the external programmer communicating with the controller of the IMD to interact with the analysis module.

18. The system of claim 11, wherein the sensor constitutes an accelerometer within the IMD.

19. The system of claim 11, wherein the analyzing module analyzes at least one of an energy or time delay in the split S2 heart sound as the characteristic of interest, the energy or time delay in the split S2 heart sound changing as the pacing parameter is changed; and wherein the setting module sets a desired value for the pacing parameter based on at least one of the energy or time delay in the split S2 heart sound.

20. The system of claim 11, wherein the pacing parameter represents at least one of an AV delay, a VV delay and a VA delay, atrial and ventricular electrode combinations for pacing, atrial and ventricular electrodes to use for sensing, timing delays between left ventricular electrodes, time delays between left atrial electrodes, timing delays between LA and LV electrodes.
